SEQUENCE LISTING (21 sequences)

SEQ ID NO: 1 - protein, 366 aa, Rhizopus oryzae
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 2 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVLDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQRESRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGSCL

SEQ ID NO: 3 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKMIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 4 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSYGFLHPGVESWIKSGTSNVQICTSEIETKDCSNSIVPFTSILDHLSYFGINEGSCL

SEQ ID NO: 5 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDEQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 6 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYNVIVTGHSLGGAQALLAGMDLYQREPRLSPKNLSIFTVGGPRVGNPTFAYYVESTGIPFQRTVHKRDIVPHVPPQSFGFLHPGVESWIKSGTSNVQICTSEIETKDCSNSIVPFTSILDHLSYFDINEGSCL

SEQ ID NO: 7 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSGTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKVAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVHKRDIVPHVPQQSFGFLHPGVESWIKSGTSNVQICTSEIETKDCSNSIVPFTSILDHLSYFDINEGSCL

SEQ ID NO: 8 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGSCL

SEQ ID NO: 9 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVLDGKIITTFTSPLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQRESRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGSCL

SEQ ID NO: 10 - protein, 366 aa, Artificial Sequence
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVLDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQRESRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGFCL

SEQ ID NO: 11 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVLDGKIITTFTSLLSDTNGYVLRSDKQKTIYLVFRGTNSFRSAITDIVFNFSDYKPVKGAKVHAGFLSSYEQVVNDYFPVVHEQLTAHPTYKVIVTGHSLGGAQALLAGMDLYQRESRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGSCL

SEQ ID NO: 12 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVLDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQRESRLSPKNLSIFTVGGPRVGNPTFAYYVESTGIPFQRTVNKRDIVPHVPSQSFGFLHPGVESWIKSGTSNVQICTSEIETKDCSNSIVPFTSILDHLSYFDINEGSCL

SEQ ID NO: 13 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVLDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQRESRLSPKNLSIFTVGGPRVGNPTFAYYVESTGIPFQRTVNKRDIVPHVPPQSFGFLHPGVESWIKSGTSDVQICTSEIETKDCSNSIVPFTSILDHLSYFDINEGSCL

SEQ ID NO: 14 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSPLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGSCL

SEQ ID NO: 15 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGFCL

SEQ ID NO: 16 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVHEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGSCL

SEQ ID NO: 17 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPSQSFGFLHPGVESWIKSGTSNVQICTSEIETKDCSNSIVPFTSILDHLSYFDINEGSCL

SEQ ID NO: 18 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSDVQICTSEIETKDCSNSIVPFTSILDHLSYFDINEGSCL

SEQ ID NO: 19 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFEFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSILDHLSYFDINEGFCL

SEQ ID NO: 20 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFNFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSITDHLSYFDINEGFCL

SEQ ID NO: 21 - protein, 366 aa, Artificial
  Mutant of Rhizopus oryzae lipase
VPVSGKSGSSNTAVSASDNAALPPLISSRCAPPSNKGSKSDLQAEPYNMQKNTEWYESHGGNLTSIGKRDDNLVGGMTLDLPSDAPPISLSSSTNSASDGGKVVAATTAQIQEFTKYAGIAATAYCRSVVPGNKWDCVQCQKWVPDGKIITTFTSLLSDTNGYVLRSDKQKTIYLVFRGTNSFRSAITDIVFEFSDYKPVKGAKVHAGFLSSYEQVVNDYFPVVQEQLTAHPTYKVIVTGHSLGGAQALLAGMDLYQREPRLSPKNLSIFTVGGPRVGNPTFAYYVESTGIPFQRTVNKRDIVPHVPPQSFGFLHPGVESWIKSGTSNVQICTSEIETKDCSNSIVPFTSITDHLSYFDINEGFCL

(19) United States
(12) Patent Application Publication
(10) Pub. No.: US 2020/0002691 A1
(43) Pub. Date: Jan. 2, 2020

(54) LIPASES WITH INCREASED THERMOSTABILITY

(71) Applicant: Henkel AG & Co. KGaA, Duesseldorf (DE)

(72) Inventors: NINA MUSSMANN, Willich (DE); SUSANNE WIELAND, Zons/Dormagen (DE); DANIELA HERBST, Duesseldorf (DE); MARGRET VAN LIER, Hilden (DE); ROLAND WEIS, Kumberg (AT)

(73) Assignee: Henkel AG & Co. KGaA, Duesseldorf (DE)

(21) Appl. No.: 16/484,909

(22) PCT Filed: Jan. 29, 2018
(86) PCT No.: PCT/EP2018/052035

§ 371 Date: Aug. 9, 2019

(30) Foreign Application Priority Data
Feb. 9, 2017 (DE) 10 2017 202 034.2

(51) Int. Cl. C12N 9/20

(52) U.S. Cl. CPC C12N 9/20

(57) ABSTRACT

The Present disclosure relates to lipases comprising an amino acid sequence which has at least 70% sequence identity with the amino acid sequence specified in SEQ ID NO:1 over the entire length thereof and which have an amino acid substitution in at least one of the positions S93, P145, L156, K169, N193, G202, Q225, K235, V236, P260, H298, P308, F311, N328, L352, D359 or S364, referring in each case to the numbering according to SEQ ID NO:1, and the production and use thereof. Lipases of such kind have very good stability, particularly thermal stability, and at the same time good cleaning power.

### CROSS-REFERENCE TO RELATED APPLICATION

This application is a U.S. National-Stage entry under 35 U.S.C. § 371 based on International Application No. PCT/EP2018/052035, filed Jan. 29, 2018, which was published under PCT Article 21(2) and which claims priority to German Application No. 10 2017 202 034.2, filed Feb. 9, 2017, which are all hereby incorporated in their entirety by reference.

### TECHNICAL FIELD

The present disclosure is directed to the field of enzyme technology. The present disclosure relates to lipases from Rhizopus oryzae in which the amino acid sequence has been modified, particularly for the purpose of using them in detergents and cleaning agents, to lend them better thermal stability, and the nucleic acids which code for them, and production thereof. The present disclosure further relates to uses of said lipases and processes in which they are used and agents which contain them, particularly detergents and cleaning agents.

### BACKGROUND

Lipases are among the most important enzymes known for technical applications. Their use in detergents and cleaning agents is firmly established in industry and they are contained in practically all modern, powerful detergents and cleaning agents. Lipases are enzymes which catalyse the hydrolysis of ester bonds in lipid substrates, particularly in greases and oils, and thus belong to the group of esterases. Lipases are typically enzymes which can cleave many substrates, for example aliphatic, alicyclic, bicyclic and aromatic esters, thioesters and activated amines. Lipases are used to remove grease-containing stains by catalysing the hydrolysis (lipolysis) thereof. Lipases with broad substrate spectra particularly used where inhomogeneous raw materials or substrate mixtures must be converted, that is to say in detergents and cleaning agents, for example, since stains can include variously structured greases and oils. The lipases used in the detergents and cleaning agents known from the related art are usually of microbial origin and are most derived from bacteria or fungi, for example the species Bacillus, Pseudomonas, Acinetobacter, Micrococcus, Humicola, Trichoderma or Trichosporon. Lipases are usually produced according to the biotechnological processes known per se by suitable microorganisms, for example by transgene expression hosts of the Bacillus species or by filamentous fungi.

A lipase provided for detergents and cleaning agents from Pseudomonas sp. ATCC 21808 is disclosed in European patent application EP 443063, for example. A lipase from Rhizopus oryzae is disclosed in the Japanese patent application JP 1225490. In general, only selected lipases are even suitable for use in liquid surfactant-containing preparations. Many lipases do not have sufficient catalysing power or stability in preparations of this kind. Particularly in washing processes, which are generally carried out at temperatures above about 20° C., many lipases are thermally unstable, which in turn results in inadequate catalytic activity during the washing process. This problem area is worsened in phosphonate-containing liquid surfactant preparations, for example, because of the complex-forming properties of the phosphonates or due to unfavourable interactions between the phosphonate and the lipase.

As a result, lipase- and surfactant-containing liquid formulations from the related art have the disadvantage that in the temperature ranges required for a washing process they often do not exhibit satisfactory lipolytic activity, and their cleaning performance with regard to lipase-sensitive soiling is consequently less than optimal.

### BRIEF SUMMARY

This disclosure provides a lipase comprising an amino acid sequence which has the at least about 70% sequence identity with the amino acid sequence specified in SEQ ID NO:1 over the entire length thereof and which has an amino acid substitution in at least one of the position S93, P145, L156, K169, N193, G202, Q225, K235, V236, P260, H298, P308, F311, N328, L352, D359 or S364, referring in each case to the numbering according to SEQ ID NO:1.

This disclosure also provides a method for producing a lipase comprising substituting an amino acid in at least one of the positions corresponding to positions 93, 145, 156, 169, 193, 202, 225, 235, 236, 260, 298, 308, 311, 328, 352, 359 and 364 in SEQ ID NO:1 in a starter lipase which has at least about 70% sequence identity with the amino acid sequence specified in SEQ ID NO:1 over the entire length thereof.

This disclosure further provides a lipase obtained from the aforementioned lipase as starting molecule by single or multiple conservative amino acid substitution, wherein the lipase includes at least one of the amino acid substitutions S93G, P145L, L156P, K169E, N193E, G202V, Q225H, K235N, V236M, P260S, H298N, F311Y, N328D, L352T, D359G and S364F in the positions corresponding to positions 93, 145, 156, 169, 193, 202, 225, 235, 236, 260, 298, 308, 311, 328, 352, 359 and 364 according to SEQ ID NO:1

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Surprisingly, it has now been found that a lipase from Rhizopus oryzae or a lipase which is sufficiently similar thereto (in terms of sequence identity), which has an amino acid substitution in at least one of the positions S93, P145, L156, K169, N193, G202, Q225, K235, V236, P260, H298, P308, F311, N328, L352, D359 or S364, referring in all cases to the numbering according to SEQ ID NO:1, is improved in terms of (thermal) stability compared with the wild type form, and is therefore suitable in particular for use in the detergents or cleaning agents.

Accordingly, in a first aspect the object of the present disclosure is a lipase comprising an amino acid sequence which has at least about 70% sequence identity with the amino acid sequence specified in SEQ ID NO:1 over the entire length thereof and has an amino acid substitution in at least one of the positions S93, P145, L156, K169, N193, G202, Q225, K235, V236, P260, H298, P308, F311, N328, L352, D359 or S364, referring in all cases to the numbering according to SEQ ID NO:1.

The lipase preferably has an amino acid substitution at position H298, particularly preferably the amino acid substitution H298N. In a particularly preferred object of the present disclosure, in addition to the amino acid substitution at position 298 the lipase has at least one further amino acid substitution at one of the positions S93, P145, L156, K169, N193, G202, Q225, K235, V236, P260, H298, P308, F311, N328, L352, D359 or S364. Particularly preferred is an amino acid substitution from the group including S93G, P145L, L156P, K169E, N193E, G202V, Q225H, K235N, V236M, P260S, H298N, P308S, P308T, F311Y, N328D, L352T, D359G and S364F referring in all cases to the numbering according to SEQ ID NO:1.

Most particularly preferably, the lipase has at least two amino acid substitutions at positions H298 and S364, the amino acid substitutions are particularly preferably H298N and S364F, referring in both cases to the numbering according to SEQ ID NO:1.

A further object of the present disclosure is a method for producing a lipase comprising the substitution of an amino acid in at least one position that corresponds to the position S93, P145, L156, K169, N193, G202, Q225, K235, V236, P260, H298, P308, F311, N328, L352, D359 or S364 in SEQ ID NO:1 in a starting lipase which has at least 70% sequence identity with the amino acid sequence specified in SEQ ID NO:1 over the entire length thereof, in such manner that the lipase has at least one of the amino acid substitutions S93G, P145L, L156P, K169E, N193E, G202V, Q225H, K235N, V236M, P260S, H298N, P308S, P308T, F311Y, N328D, L352T, D359G and S364F.

A method for producing a lipase comprising the substitution of an amino acid in at least position H298 is preferred, the amino acid substitution is particularly preferably H298N.

Most particularly preferred is a method for producing a lipase comprising the substitution of an amino acid in at least the two positions H298 and S364, the amino acid substitutions are particularly preferably H298N and S364F, each referring to the numbering according to SEQ ID NO:1.

For the purposes of the present patent application, a lipase therefore comprises both the lipase as such and also a lipase produced in a method as contemplated herein. All explanations regarding the lipase therefore refer both to the lipase as such and to the lipases that are produced by employing corresponding methods.

Further aspects of the present disclosure relate to the nucleic acids that code for these lipases, non-human host cells containing lipases or nucleic acids as contemplated herein and media comprising lipases as contemplated herein, particularly detergents and cleaning agents, washing and cleaning processes and uses of the lipases as contemplated herein in detergents or cleaning agents to remove greasy soiling.

As used in this document, the phrase “at least one” signified one or more, i.e. 1, 2, 3, 4, 5, 6, 7, 8, 9, 10, 11, 12, 13, 14 or more.

The present disclosure is based on the surprising discovery that an amino acid substitution in at least one of the positions 93, 145, 156, 169, 193, 202, 225, 235, 236, 260, 298, 308, 311, 328, 352, 359 or 364 of the lipase from Rhizopus oryzae according to SEQ ID NO:1, in a lipase which comprises an amino acid sequence that is at least about 70% identical to the amino acid sequence specified in SEQ ID NO:1 in such manner that the amino acids S93, P145, L156, K169, N193, G202, Q225, K235, V236, P260, H298, P308, F311, N328, L352, D359 or S364 are present in at least one of the corresponding positions, has the effect of improving (thermal) stability to this modified lipase in detergents and cleaning agents. This is particularly surprising since none of the aforementioned amino acid substitutions has been associated with increased stability of the lipase previously.

The lipases as contemplated herein manifest greater stability in detergents or cleaning agents, particularly when exposed to higher temperatures. Such performance-enhanced lipases enable improved washing results for lipolytically sensitive soiling in a broad temperature range.

The lipases as contemplated herein demonstrate enzymatic activity, which means that they are able to hydrolyse greases and oils, particularly in a detergent or cleaning agent. A lipase as contemplated herein is therefore an enzyme which catalyses the hydrolysis of ester bonds in lipid substrates, and is thus capable of cleaving greases or oils. A lipase as contemplated herein is also preferably a mature lipase, i.e. the catalytically active molecule with no signal and/or propeptide(s). Unless indicated otherwise, the specified sequences also refer to mature (processed) enzymes in each case.

In various embodiments, the lipase as contemplated herein contains at least one amino acid substitution, selected from the group including S93G, P145L, L156P, K169E, N193E, G202V, Q225H, K235N, V236M, P260S, H298N, P308S, P308T, F311Y, N328D, L352T, D359G and S364F, each referring to the numbering according to SEQ ID NO:1. In further preferred embodiments, the lipase as contemplated herein contains one of the following amino acid substitution variants: (i) P145L, P260S and H298N; (ii) V236M; (iii) F311Y and D359G; (iv) K169E; (v) K235N; (vi) S93G, G202V and P308Q; (vii) H298N; (viii) P145L, P260S, H298N and L156P; (ix) P145L, P260S, H298N and S364F; (x) P145L, P260S, H298N and Q225H; (xi) P145L, P260S, H298N and P308S; (xii) P145L, P260S, H298N and N328D; (xiii) H298N and L156P; (xiv) H298N and S364F; (xv) H298N and Q225H; (xvi) H298N and P308S; (xvii) H298N and N328D; (xviii) H298N, S364F and N193E; (xix) H298N, S364F and L352T; or (xx) H298N, S364F, N193E and L352T, wherein the numbering refers to the numbering according to SEQ ID NO:1 in each case. In other embodiments, the variants that contain a substitution in position 298, particularly 298N are preferred. Of these, the variants which do not have any substitutions in positions 145 and 260 are particularly preferred. Most preferred as those which have a substitution in position 298, particularly 298N, and at least one substitution in one of the positions 156, 225, 308, 328 or 364, but no substitution in positions 145 and 260. This applies particularly for the abovementioned variants (xiv) to (xx).

In a further embodiment of the present disclosure, the lipase comprises an amino acid sequence which is at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5% and about 98.8% identical with the amino acid sequence specified in SEQ ID NO:1 over the entire length thereof and has one or more of the amino acid substitutions in at least one of the positions 93, 145, 156, 169, 193, 202, 225, 235, 236, 260, 298, 308, 311, 328, 352, 359 or 364, particularly in at least one of the positions S93, P145, L156, K169, N193, G202, Q225, K235, V236, P260, H298, P308, F311, N328, L352, D359 or S364, more preferably at least one of the substitutions S93G, P145L, L156P, K169E, N193E, G202V, Q225H, K235N, V236M, P260S, H298N, P308S, P308T, F311Y, N328D, L352T, D359G and S364F in the numbering according to SEQ ID NO. 1. In the context of the present disclosure, this features means that the lipase has the specified substitutions, that it contains at least one of the corresponding amino acids in the corresponding positions, i.e., not all of the 13 positions are otherwised mutated or deleted by fragmentation of the lipase, for example. The amino acid sequences of such lipases, which are preferred as contemplated herein, are indicated in SEQ ID Nos: 2-21.

The identity of nucleic acid or amino acid sequences is determined by sequence comparison. Said sequence comparison is based on the BLAST algorithm (see for example Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of protein database search programs”; Nucleic Acids Res., 25, S.3389-3402), which is recognised and used regularly in the related art, and functions in principle by assigning similar nucleotides or amino acids in the nucleic acid or amino acid sequences to each other. An assignment of the positions concerned in table form is called an alignment. Another algorithm which is available in the related art is the FASTA algorithm. Sequence comparisons (alignments), particularly multiple sequence comparisons, are created with computer programs. Use is often made of the Clustal series (see for example Chenna et al. (2003): Multiple sequence alignment with the Clustal series of programs. Nucleic Acid Research 31, 3497-3500), T-Coffee (see for example Notredame et al. (2000): T-Coffee: A novel method for multiple sequence alignments. J. Mol. Biol. 302, 205-217) for example, or programs based on these programs and algorithms. Sequence comparisons (alignments) with the software program Vector NTI® Suite 10.3 (Invitrogen Corporation, 1600 Faraday Avenue, Carlsbad, Calif., USA) with the prescribed standard parameters, whose AlignX module for sequence comparisons is based on ClustalW is also possible. Unless otherwise indicated, the sequence identity specified in this document is determined with the BLAST algorithm.

Such a comparison also enables a statement to be made about the similarity of the compared sequences to each other. It is usually expressed as percent identity, that is to say as the percentage of identical nuceotides or amino acid residues thereof, or in an alignment of corresponding positions. With regard to amino acid sequences, the more broadly defined notion of homology also extends to conservative amino acid replacements, that is to say amino acids with similar chemical activity, since these usually perform similar chemical activities within the protein. Thus, the similarity of the compared sequences may also be expressed as percent homology or percent similarity. Values for identity and/or homology may be calculated for entire polypeptides or genes or only for individual regions. Homologous or identical regions of different nucleic acid or amino acid sequences are therefore defined by matches in the sequences. Such regions often have identical functions. They can be small, involving only a small number of nucleotides or amino acids. Such small regions often perform essential functions for the overall activity of the protein. It may therefore be advisable to relate sequence matches only to individual, possibly small regions. Unless otherwise indicated, however, identity or homology values given in the present patent application refer to the total length of the specified nucleic acid or amino acid sequence in each case.

In the context of the present disclosure, the statement that an amino acid position corresponds to a numerically described position in SEQ ID NO:1 therefore means that the corresponding numerically described position in SEQ ID NO:1 is assigned in an alignment as defined earlier.

In a further embodiment of the present disclosure, the lipase has cleaning power that is not significantly reduced compared with that of a lipase comprising an amino acid sequence which corresponds to the amino acid sequence specified in SEQ ID NO:1, i.e., that it has at least about 80%, preferably at least about 100%, more preferably yet at least about 110% of the reference washing power. The cleaning power may be determined in a washing system which contains a detergent in a measured quantity between about 4.5 and about 7.0 gram per litre of washing liquor and the lipase, wherein the lipases for comparison are used in identical concentrations (relative to active protein) and the cleaning power against soiling on cotton is determined by measuring the degree of cleaning of the washed textiles. For example, the washing process may be performed for about 70 minutes at a temperature of about 40° C. and the water may have a water hardness between about 15.5 and about 16.5° (German hardness). The concentration of the lipase in the detergent investigated for this washing system is from about 0.001-0.1 percent by weight, preferably from about 0.01 to about 0.06 percent by weight relative to active, cleaned protein.

A liquid reference detergent for such a washing system may be composed as follows (all values in percent by weight): about 7% alkylbenzene sulfonic acid, about 9% other anionic surfactants, about 4% C12-C18 Na-salts of fatty acids (soaps), about 7% non-ionic surfactants, about 0.7% phosphonated, about 3.2% citric acid, about 3.0% NaOH, about 0.04% defoaming agents, about 5.7% 1,2-propanediol, about 0.1% preservatives, about 2% ethanol, about 0.2% dye transfer inhibitor, the rest being demineralised water. The measured quantity of the liquid detergent preferably amounts to between about 4.5 and about 6.0 gram per litre washing liquor, for example about 4.7, about 4.9 or about 5.9 gram per litre washing liquor. Washing is preferably carried out in a pH value range between about pH 8 and about pH 10.5, preferably between about pH 8 and about pH 9.

For the purposes of the present disclosure, the determination of cleaning power is carried out for example at about 34.8° C. using a liquids detergent such as was described earlier, wherein the washing process preferably lasts for about 30 minutes.

The degree of whiteness, i.e. the degree by which the soiling is brightened as a measure of cleaning power, is determined using optical measurement methods, preferably photometrically. One device that is suitable for this is for example the Minolta CM508d spectrometer. The devices used for the measurement are usually calibrated beforehand with a white standard, preferably a white standard supplied with the device.

The use of the respective lipase for the same activity ensures that even if there is some divergence in the ratio of active substance to total protein (the values of the specific activity) the respective enzymatic properties—that is to say for example the cleaning power in respect of certain types of soiling—are still compared. In general, a low specific activity can be compensated for by adding a larger quantity of protein.

Otherwise the lipase activity may also be determined in the manner commonly accepted in the art, and preferably as described in Bruno Stellmach, “Bestimmungsmethoden Enzyme für Pharmazie, Lebensmittelchemie, Technik, Biochemie, Biologie, Medizin [Methods for determining enzymes for pharmaceuticals, food chemistry, technology, biochemistry, biology, medicine]” (Steinkopff Verlag Darmstadt, 1988, p. 172ff). In this method, lipase-containing samples are added to an olive oil emulsion in emulsifier-containing water and incubated at about 30° C. and about pH 9.0. This releases fatty acids. The fatty acids are titrated continuously for about 20 minutes with about 0.01 N sodium hydroxide solution in an autotitrator, so that the pH value remains constant (“pH stat titration”). The lipase activity is determined on the basis of the consumption of sodium hydroxide solution by comparing to a reference lipase sample.

An alternative test to determine lipolytic activity of the lipases as contemplated herein is an optical measurement method, preferably a photometric method. The test that is suitable for this comprises lipase-dependent cleavage of the substrate para-nitrophenyl-butyrate (pNP-butyrate). This substrate is split into para-nitrophenolate and butyrate. The presence of para-nitrophenolate can be determined using a photometer, e.g., the Tecan Sunrise device and XFLUOR software at 405 nm, thereby enabling a conclusion to be drawn about the enzymatic activity of the lipase.

The protein concentration can be determined with the aid of known methods, for example the BCA assay (bicinchoninic acid; 2,2′-biquinoline-4,4′-dicarboxylic acid) or the biuret test (A. G. Gornall, C. S. Bardawill and M. M. David, J. Biol. Chem., 177 (1948), p. 751-766). In this context, determination of the active protein concentration can be carried out by titration of the active centres using a suitable irreversible inhibitor and determining the residual activity (see M. Bender et al., J. Am. Chem. Soc. 88, 24 (1966), p. 5890-5913).

Besides the amino acid modifications explained previously, lipases as contemplated herein may also have further amino acid modifications, particularly amino acid substitutions, insertions or deletions. Such lipases are developed further, for example by selective genetic alteration, i.e. mutagenesis methods, and optimised for specific purposes or in respect of special properties (for example with a view to their catalytic activity, stability, etc.). Nucleic acids as contemplated herein may also be introduced into recombination formulas and thus used to generate entirely novel lipases or other polypeptides.

The objective is to introduce targeted mutations such as substitutions, insertions or deletions into the known molecules to improve the cleaning power of enzymes as contemplated herein, for example. For this purpose, particularly the surface charges and/or the isoelectric points of the molecules and consequently their interactions with the substrate may be modified. In this way, for example the net charge of the enzymes may be changed so that through them the substrate bond may be influenced, particularly for use in detergents and cleaning agents. Alternatively or in addition thereto the stability of the lipase may be increased still further by one or more corresponding mutations, thereby improving their cleaning power. Advantageous properties of individual mutations, e.g., individual substitutions, may complement each other. A lipase which has already been optimised in respect of certain properties, for example in terms of its stability when exposed to higher temperatures, may thus be enhanced further within the scope of the present disclosure.

In order to describe substitutions which affect exactly one amino acid position (amino acid replacements), the following convention is applied in this application: first, the amino acid that is naturally present is designated in the form of the commonly used international single letter code, then follows the associated sequence position and finally the inserted amino acid. Multiple replacements within the same polypeptide chain are separated from each other by forward slashes. In the case of insertions, additional amino acids are named after the sequence position. For deletions, the missing amino acid is replaced with a symbol, an asterisk or hyphen for example, or a Δ is placed before the corresponding position. For example, H298N describes the substitution of histidine in position 298 by asparagine, H298HE the insertion of glutamic acid after the amino acid histidine in position 298, and H298* or ΔH298 represents the deletion of histidine in position 298. This nomenclature is familiar to the person skilled in the field of enzyme technology.

A further object of the present disclosure is therefore a lipase which may be obtained from a lipase as the starting molecule as described earlier by single or multiple conservative amino acid substitution, wherein the lipase in the numbering according to SEQ ID NO:1 still has at least one of the amino acid substitutions as contemplated herein in the positions corresponding to positions 93, 145, 156, 169, 193, 202, 225, 235, 236, 260, 298, 308, 311, 328, 352, 359 and 364 in SEQ ID NO:1, as described earlier. The term “conservative amino acid substitution” means the replacement (substitution) of one amino acid residue with another amino acid residue, wherein this replacement does not result in a change in the polarity or charge in the position of the substituted amino acid, e.g., the replacement of a non-polar amino acid residue with another non-polar amino acid residue. For the purposes of the present disclosure, conservative amino acid substitutions include for example: G=A=S, I=V=L=M, D=E, N=Q, K=R, Y=F, S=T, G=A=I=V=L=M=Y=F=W=P=S=T.

Alternatively or additionally, the lipase may be obtained from the lipase as contemplated herein as the starting molecule by fragmentation, deletion, insertion or substitution mutagenesis and that it includes an amino acid sequence which matches the starting molecule over a length of at least about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360, about 361, about 362, about 363, about 364 or about 365 consecutive amino acids, wherein the amino acid substitution(s) obtained in the starting molecule is/are still present in one or more of the positions corresponding to positions 93, 145, 156, 169, 193, 202, 225, 235, 236, 260, 298, 308, 311, 328, 352, and 359 and 364 in SEQ ID NO:1.

Thus it is possible for example to delete individual amino acids at the termini or in the loops of the enzyme without thereby losing or reducing lipolytic activity. Moreover, fragmentation, deletion, insertion or substitution mutagenesis of such kind may also serve for example to lower the allergenicity of the enzymes in question, which in turn improves their general usability. The enzyme advantageously retain their lipolytic activity even after mutagenesis, i.e. their lipolytic activity is at least equal to that of the starter enzyme, that is to say the lipolytic activity in a preferred embodiment is equal to at least about 80, preferably at least about 90% of the activity of the starter enzyme. Further substitutions may also have advantageous effects. Both individual and multiple consecutive amino acids may be replaced with other amino acids.

In various embodiments, the lipases of the present disclosure are C-terminal fragments of the proteins described herein. Such fragments are particularly preferably those in which the N-terminal prosequence, i.e. the first 69 amino acids of the sequence according to SEQ ID NO:1 are missing. All of the sequences described in the present application may be corresponding fragments, in which the amino acids corresponding to amino acids from about 1-69 of the lipase with the sequence according to SEQ ID NO:1 are missing. This applies particularly for the mutants described herein with SEQ ID Nos. 2-21. The correspondingly mature sequences, in which the first 69 amino acids of the sequences according to SEQ ID Nos. 2-21 are missing are also explicitly included herewith. In different embodiments, the present disclosure therefore also relates to lipases which comprise amino acids from about 70-366 of the amino acid sequences according to SEQ ID nos. 2-21 or consist thereof. In different embodiments of the present disclosure, this therefore also includes lipases which have about 70% sequence identity with amino acids from about 70-366 of the amino acid sequence specified in SEQ ID NO:1 and an amino acid substitution in at least one of the positions S93, P145, L156, K169, N193, G202, Q225, K235, V236, P260, H298, P308, F311, N328, L352, D359 or S364, referring in each case to the numbering according to SEQ ID NO:1. All of the embodiments disclosed in the context herein with the lipases containing the propeptide may also be transferred to the corresponding mature lipases, in which the sequence corresponding to the propeptide, which corresponds to amino acids 1-69 according to SEQ ID NO:1 is missing.

Also included are variants which are C- and/or N-terminal extended compared with the variants described herein, that is to say variants which comprise from about 1 to about 68 additional amino acids at the N- and/or C-terminus, for example. N-terminal extended variants are for example the previously described mature variants of the polypeptide sequences (comprising amino acids from about 70-366) specified in the SEQ ID nos. 2-21, which also comprise residues of the originally about 69 amino acid long prosequence.

Alternatively or additionally, the lipase can be obtained from a lipase as contemplated herein as starting molecule by single or multiple conservative amino acid substitution, wherein the lipase includes at least one of the amino acid substitutions S93G, P145L, L156P, K169E, N193E, G202V, Q225H, K235N, V236M, P260S, H298N, P308S, P308T, F311Y, N328D, L352T, D359G and S364F in the positions which correspond to positions 93, 145, 156, 169, 193, 202, 225, 235, 236, 260, 298, 308, 311, 328, 352, and 359 and 364 according to SEQ ID NO:1.

In further embodiments, the lipase can be obtained from a lipase as contemplated herein as starting molecule by fragmentation, deletion, insertion or substitution mutagenesis and comprises an amino acid sequence which matches the starting molecule over a length of at least about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360 or about 366 consecutive amino acids, wherein the lipase comprises at least one of the amino acid substitutions S93G, P145L, L156P, K169E, N193E, G202V, Q225H, K235N, V236M, P260S, H298N, P308S, P308T, F311Y, N328D, L352T, D359G and S364F in the positions corresponding to positions 93, 145, 156, 169, 193, 202, 225, 235, 236, 260, 298, 308, 311, 328, 352, and 359 and 364 according to SEQ ID NO:1.

In this context, the further amino acid positions are defined by an alignment of the amino acid sequence of a lipase as contemplated herein with the amino acid sequence of the lipase from Rhizopus oryzae, as specified in SEQ ID NO:1. The assignment of the positions also conforms to the mature protein. This assignment must also be applied particularly when the amino acid sequence of a lipase as contemplated herein comprises a larger number of amino acid residues than the lipase from Rhizopus oryzae according to SEQ ID NO. 1. Starting from the cited positions in the amino acid sequence of the lipase from Rhizopus oryzae, the modification positions in a lipase as contemplated herein are precisely those which are assigned to these positions in an alignment.

Accordingly, advantageous positions for sequence modifications, particularly substitutions, of the lipase from Rhizopus oryzae, which are preferably significant when transferred to homologous positions of the lipases as contemplated herein and which lend the lipase advantageous functional properties, are the positions in an alignment which correspond to positions 93, 145, 156, 169, 193, 202, 225, 235, 236, 260, 298, 308, 311, 328, 352, and 359 and 364 in SEQ ID NO:1, i.e. in the numbering according to SEQ ID NO:1. The cited positions in the wild type molecule of the lipase from Rhizopus oryzae are occupied by the following amino acid residues: S93, P145, L156, K169, N193, G202, Q225, K235, V236, P260, H298, P308, F311, N328, L352, D359 or S364.

Further confirmation of the correct assignment of the amino acids to be modified, i.e. particularly their functional correspondence, may be provided by comparative tests, according to which the two positions assigned to each other on the basis of an alignment are modified in the same way in both of the lipases that are compared and observations are carried out to determine whether the enzymatic activity is modified in the same way for both. For example, if an amino acid replacement in a given position of the lipase from Rhizopus oryzae according to SEQ ID NO:1 is associated with a change in an enzymatic parameter, for example with increase of the KM value, and if a corresponding change in the enzymatic parameter, for example an increase of the KM value, is also observed in a lipase variant as contemplated herein whose amino acid replacement was effected by the same introduced amino acid, this may be considered confirmation of the correct assignment.

All of the information presented is also applicable to the methods as contemplated herein for preparing a lipase. Accordingly, a method as contemplated herein further comprises one or more of the following method steps:

a) Introducing a single or multiple conservative amino acid substitution, wherein the lipase comprises at least one of the amino acid substitutions S93G, P145L, L156P, K169E, N193E, G202V, Q225H, K235N, V236M, P260S, H298N, P308S, P308T, F311Y, N328D, L352T, D359G and S364F in the positions which correspond to positions 93, 145, 156, 169, 193, 202, 225, 235, 236, 260, 298, 308, 311, 328, 352, 359 and 364 according to SEQ ID NO:1;
b) Modifying the amino acid sequence by fragmentation, deletion, insertion or substitution —mutagenesis in such manner that the lipase comprises an amino acid sequence, which matches the starting molecule over a length of at least about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 270, about 280, about 290, about 300, about 310, about 320, about 330, about 340, about 350, about 360 or about 366 consecutive amino acids, wherein the lipase comprises at least one of the amino acid substitutions S93G, P145L, L156P, K169E, N193E, G202V, Q225H, K235N, V236M, P260S, H298N, P308S, P308T, F311Y, N328D, L352T, D359G and S364F in the positions which correspond to positions 93, 145, 156, 169, 193, 202, 225, 235, 236, 260, 298, 308, 311, 328, 352, 359 and 364 according to SEQ ID NO:1.

All notes are also valid for the methods as contemplated herein.

In further variants of the present disclosure, the lipase and/or the lipase prepared with a method as contemplated herein is also at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, or about 98.8% identical with the amino acid sequence specified in SEQ ID NO:1 over the entire length thereof. Alternatively, the lipase and/or the lipase prepared with a method as contemplated herein is also at least about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, or about 98% identical to one of the amino acid sequences specified in SEQ ID nos:2-10 or 11-21 over the entire length thereof. The lipase and/or the lipase prepared with a method as contemplated herein has an amino acid substitution in at least one of the positions S93, P145, L156, K169, N193, G202, Q225, K235, V236, P260, H298, P308, F311, N328, L352, D359 or S364, referring in all cases to the numbering according to SEQ ID NO:1. In more preferred embodiments, the amino acid substitution is at least one selected from the group including S93G, P145L, L156P, K169E, N193E, G202V, Q225H, K235N, V236M, P260S, H298N, P308S, P308T, F311Y, N328D, L352T, D359G and S364F, referring in all cases to the numbering according to SEQ ID NO:1. In further preferred embodiments, the lipase comprises one of the following amino acid substitution variants: (i) P145L, P260S and H298N; (ii) V236M; (iii) F311Y and D359G; (iv) K169E; (v) K235N; (vi) S93G, G202V and P308Q; (vii) H298N; (viii) P145L, P260S, H298N and L156P; (ix) P145L, P260S, H298N and S364F; (x) P145L, P260S, H298N and Q225H; (xi) P145L, P260S, H298N and P308S; (xii) P145L, P260S, H298N and N328D; (xiii) H298N and L156P; (xiv) H298N and S364F; (xv) H298N and Q225H; (xvi) H298N and P308S; (xvii) H298N and N328D; (xviii) H298N, S364F and N193E; (xix) H298N, S364F and L352T; or (xx) H298N, S364F, N193E and L352T.

A further object of the present disclosure is a lipase described previously which is stabilised further, particularly by one or more mutations, for example substitutions, or by coupling to a polymer. Increasing stability during storage and/or during use, in the washing process for example, has the effect of prolonging the enzymatic activity and thereby improving the cleaning power. In principle, all stabilising options that are described in the related art and/or can be applied practically are suitable candidates. Preferred are those stabilising agents which are created via mutations of the enzyme itself, since such stabilising agents do not require further work steps following recovery of the enzyme. Examples of sequence modifications that are suitable for such were identified previously. Further suitable sequence modifications are known from the related art.

Capabilities of the stabilising agent are for example:
Protection against the influence of denaturing agents such as surfactants by mutations which cause a change in the amino acid sequence at or on the surface of the protein;
Replacement of amino acids located close to the N-terminus with such that enter into contact with the rest of the molecule via presumably non-covalent interactions and so contribute to the preservation of the globular structure.

Preferred embodiments are those in which the enzyme is stabilised in several ways, since multiple stabilising mutations have a cumulative or synergistic effect.

A further object of the present disclosure is a lipase as described previously, which has at least one chemical modification. A lipase with such a modification is called a derivative, meaning that the lipase has been derivatised.

Accordingly, for the purposes of the present application, derivatives are understood to be proteins of which the pure amino acid chain has been chemically modified. Such derivatisations may be carried out for example in vivo by the host cell which expresses the protein. In this context, couplings of low-molecular compounds such as lipids or oligosaccharides are particularly noteworthy. But derivatisations can also be performed in vitro, for example by the chemical conversion of an amino acid side chain or by covalent bonding of another compound to the protein. For example, the coupling of amines to carboxyl groups of an enzyme to change the isoelectric point is possible. Another such compound may also be another protein, which is bound to a protein as contemplated herein for example via bifunctional chemical compounds. Derivatisation is also understood to mean covalent bonding to a macromolecular carrier, or also non-covalent inclusion in suitable macromolecular cage structures. For example, derivatisations can influence substrate specificity or the strength of the bond with the substrate or a cause a temporary blockage of enzymatic activity if the coupled substance is an inhibitor. This may be practical for the storage period, for example. Modifications of such kind may also affect stability or enzymatic activity. Furthermore, they can also be used to lessen allergenicity and/or immunogenicity of the protein, and thereby increase its tolerability on the skin, for example. For example, couplings with macromolecular compounds such as polyethylene glycol can improve the protein in terms of stability and/or skin tolerability.

In the broadest sense, derivatives of a protein as contemplated herein may also be understood to include preparations of such proteins. Depending on how it is recovered, processed or prepared, a protein may be combined with various other substances, from the culture of the producing microorganisms, for example. Other substances may also have been added to the protein specifically to increase its storage stability, for example. Therefore, all preparations of a protein as contemplated herein fall within the scope of the present disclosure. This is also irrespective of whether it actually exhibits this enzymatic activity in a given preparation or not. Because it may be desirable for the protein to exhibit little or no activity while it is stored, and only begins to perform its enzymatic function when it is actually in use. This can be controlled with suitable admixtures, for example. The joint preparation of lipases with specific inhibitors in particular is possible in this connection.

Of all the lipases and lipase variants and/or derivatives described in the preceding text, those whose stability and/or activity corresponds to at least one of the lipases according to SEQ ID nos: 2-21 and/or whose cleaning power is equal to at least one of the lipases according to SEQ ID Nos: 2-21, wherein the cleaning power is determined in a washing system as described earlier, are particularly preferred in the context of the present disclosure.

A further object of the present disclosure is a nucleic acid which codes for a lipase as contemplated herein a vector that contains such a nucleic acid, particularly a cloning vector or an expression vector.

These may be DNA or RNA molecules. They may exist as a single strand, a single strand that complements this single strand, or as a double strand. Particularly in the case of DNA molecules, the sequences of both complementary strands must be taken into account in each of the three possible reading frames. It must also be borne in mind that different codons, i.e. base triplets, can code for the same amino acids, with the result that a certain amino acid sequence may be coded by several different nucleic acids. In consequence of this degeneracy of the genetic code, all nucleic acid sequences that are able to code one of the previously described lipases are subsumed into this object of the present disclosure. The person skilled in the art is able to determine these nucleic acid sequences beyond doubt, because defined amino acids can be assigned to individual codons despite the degeneracy of the genetic code. Consequently, the person skilled in the art has no difficulty in determining nucleic acids that code for an amino acid sequence based on that amino acid sequence. Moreover, in the case of nucleic acids as contemplated herein, one or more codon(s) may have been replaced by synonymous codons. This aspect relates in particular to the heterologous expression of the enzymes as contemplated herein. Thus, each organism—for example a host cell of a production strain—has a certain codon use. Codon use is understood to mean the transfer of genetic code to amino acids by the respective organism. Bottlenecks in protein biosynthesis may occur if the codons located on the nucleic acid are matched by a relatively small number of charged tRNA molecules. Although coding for the same amino acid, the result of this is that a codon is translated less efficiently in the organism than a synonymous codon which codes for the same amino acid. The presence of a larger number of tRNA molecules for the synonymous codon means that the codon can be translated more efficiently in the organism.

A person skilled in the art is able to apply methods which are generally known today, such as chemical synthesis or polymerase chain reaction (PCR) in conjunction with standard molecular-biological and/or protein-chemical methods, to produce the corresponding nucleic acids all the way up to entire genes based on known DNA- and/or amino acid sequences. Such methods are known for example from Sambrook, J., Fritsch, E. F. and Maniatis, T. 2001. Molecular cloning: a laboratory manual, 3. Edition Cold Spring Laboratory Press.

For the purposes of the present disclosure, vectors are understood to be elements including nucleic acids which contain a nucleic acid as the characterizing nucleic acid region. They are able to establish it as a stable genetic element over several generations or cell divisions in a species or cell line. Vectors are special plasmids, that is to say circular genetic elements, particularly when used in bacteria. In the context of the present disclosure, a nucleic acid as contemplated herein is cloned to form a vector. The vectors include for example those originating from bacterial plasmids, viruses or bacteriophages, or mostly synthetic vectors or plasmids with elements of diverse origins. With the further genetic elements present in each case, vectors are able to establish themselves as stable units in the host cells concerned over several generations. They may exist extrachromosomally as independent units or they may integrate in a chromosome or chromosomal DNA.

Expression vectors comprise nucleic acid sequences which enable them to replicate themselves in the host cells that contain them, preferably microorganisms, particularly preferably bacteria, and to bring the nucleic acid contained therein to expression. The expression is influenced particularly by the one or more promoters which regulate transcription. In principle, the expression can be carried out by the natural promoter, which was originally positioned before the nucleic acid that is to be expressed, but also by a promoter of the host cell supplied on the expression vector, or also by a modified or an entirely different promoter from another organism or another host cell. In the present case, at least one promoter is made available for the expression of a nucleic acid as contemplated herein and used for the expression thereof. Expression vectors may also be capable of regulation, for example by changing the conditions of cultivation or upon reaching a certain cell density of the host cells that contain them or by the addition of certain substances, particularly activators of the gene expression. One example of such a substance is the galactose derivative isopropyl-β-D-thiogalactopyranoside (IPTG), which is used as an activator of the bacterial lactose operon. Unlike expression vectors, the nucleic acid contained is not expressed in cloning vectors.

A further object of the present disclosure is a non-human host cell which contains a nucleic acid as contemplated herein or a vector as contemplated herein, or which contains a lipase as contemplated herein, particularly one which secretes the lipase into the medium surrounding the host cell. A nucleic acid as contemplated herein or a vector as contemplated herein is preferably transformed into a microorganism, which then represents a host cell as contemplated herein. Alternatively, individual components, i.e. nucleic acid parts or fragments of a nucleic acid as contemplated herein may be inserted in the host cell in such a way that the host cell resulting therefrom contains a nucleic acid as contemplated herein or a vector as contemplated herein. This approach is particularly suitable when the host cell already contains one or more components of a nucleiec acid as contemplated herein or of a vector as contemplated herein and the other components are then complemented correspondingly. Cell transformation methods are routine in the related art and are well known to the person skilled in the art. In principle, all cells, that is to say either prokaryotic or eukaryotic cells are suitable for use as host cells. Preferred are those host cells which lend themselves well to genetic manipulation, which has implications for transformation with the nucleic acid or the vector and the stable establishment thereof, for example single-cell fungi or bacteria. Preferred host cells are further exemplified by good microbiological and biotechnological manageability. This is reflected for example in ease of cultivation, high growth rates, low requirements regarding fermentation media and good production and secretion rates for foreign proteins. Preferred host cells as contemplated herein secrete the (transgenically) expressed protein into the medium surrounding the host cells. The lipases may also be modified by the cells that produce them after their production, for example by linking sugar molecules, formylations, aminations, etc. Such post-translational modifications may affect the functions of the lipase.

Further preferred embodiments are represented by host cells whose activity can be regulated by genetic regulation elements which are made available on the vector for example, but which may also be present in these cells beforehand. They can be induced to expression for example by controlled addition of chemical compounds serving as actuators, by changing the conditions of cultivation or when a certain cell density is reached. In this way, the proteins as contemplated herein may be produced economically. An example of such a compound is IPTG, as described earlier.

Preferred host cells are prokaryotic or bacterial cells. Bacteria are exemplified by short generation periods and low demands in respect of cultivation conditions. As a result, inexpensive cultivation methods or production methods may be set up. Besides, the person skilled in the art has a wealth of experience regarding bacteria in fermentation technology. Gram-negative or gram-positive bacteria may be suitable for special production for an enormous range of reasons, some of which can be determined experimentally, such as nutrient sources, product formation rate, time constraints etc.

In the case of gram-negative bacteria such as Escherichia coli for example, a multiplicity of proteins are secreted into the periplasmic space, that is to say the compartment between the two membranes which enclose the cells. This may be advantageous for special applications. In addition, gram-negative may also be constructed so that they discharge the expressed proteins not only inth the periplasmic space, but also into the medium surrounding the bacterium. On the other hand, gram-positive bacteria such as bacilli or actinomycetes for example, or other representatives of the Actinomycetales do not have an outer membrane, so secreted proteins are delivered directly into the medium surrounding the bacteria, typically the nutrient medium, from which the expressed proteins can be purified. They may be isolated from the medium directly of processed further. More-over, gram-positive bacteria are related or identical to most source organisms for technologically important enzymes and usually form comparable enzymes themselves, so they have a similar codon use and their protein synthesis apparatus is naturally set up appropriately.

Host cells as contemplated herein may have been modified in terms of the culture conditions they require, they may have other or additional selection markers, or they may express yet other or additional proteins. In particular, they may be host cells which express multiple proteins or enzymes.

In principle, the present disclosure may be applied to all microorganisms, particularly all fermentable microorganisms, particularly preferably to those of the Bacillus species, and is responsible for making the production of proteins as contemplated herein possible by the use of such microorganisms. Such microorganisms then represent host cells within the meaning of the present disclosure.

In a further embodiment of the present disclosure, the host cell is a bacterium, preferably one selected from the group of the species of Escherichia, Klebsiella, Bacillus, Staphylococcus, Corynebakterium, Arthrobacter, Streptomyces, Stenotrophomonas and Pseudomonas, more preferably one selected from the group of Escherichia coli, Klebsiella planticola, Bacillus licheniformis, Bacillus lentus, Bacillus amyloliquefaciens, Bacillus subtilis, Bacillus alcalophilus, Bacillus globigii, Bacillus gibsonii, Bacillus clausii, Bacillus halodurans, Bacillus pumilus, Staphylococcus carnosus, Corynebacterium glutamicum, Arthrobacter oxidans, Streptomyces lividans, Streptomyces coelicolor and Stenotrophomonas maltophilia.

However, the host cell may also be a eukaryotic cell, which has a nucleus. A further object of the present disclosure is therefore a host cell which has a nucleus. Unlike prokaryotic cells, eukaryotic cells are able to post-translationally modify the proteins formed. Examples of these are fungi such as Actinomycetes or yeasts such as Saccharomyces or Kluyveromyces. This may be particularly advantageous for example when the proteins are intended to undergo specific modifications associated with their synthesis which enable such systems. The modifications carried out by eukaryotic systems particularly in connection with protein synthesis include for example binding low-molecular compounds like membrane anchors or oligosaccharides. Oligosaccharide modifications of such kind may be desirable reducing the allergenicity of the expressed protein, for example. A co-expression with the enzymes formed naturally by such cells, such as cellulases, may also be advantageous. In addition, thermophilic fungal expression systems for example may also lend themselves particularly well to the expression of thermally resistant proteins or variants.

The host cells as contemplated herein are cultivated and fermented in the usual way, for example in discontinuous or continuous systems. In the former case, a suitable nutrient medium is inoculated with the host cells and the product is harvested from the medium after a period which must be determined experimentally. Continuous fermentations are notable for reaching a steady state in which a fraction of the cells dies after a relatively long period but also grow again, and the protein formed can be removed from the medium at the same time.

Host cells as contemplated herein are preferably use to produce lipases as contemplated herein. A further object of the present disclosure is therefore a method for producing a lipase, comprising

a) Cultivating a host cell as contemplated herein, and
b) Isolating the lipase from the culture medium or the host cell.

This object of the present disclosure preferably comprises fermentation processes. Fermentation processes are known from the related art and represent the actual large-scale production step, typically followed by a suitable purification method of the manufactured product, for example the lipases as contemplated herein. All fermentation processes which are based on a corresponding method for producing a lipase as contemplated herein are embodiments of this object of the present disclosure.

Fermentation processes that the fermentation is carried out with a feed strategy are particularly suitable for use. In these, the media components that are consumed by the continuous cultivation are added to the feedstock. In this way, considerable enhancements in both cell density and cell mass or dry mass, and/or particularly in the activity of the lipase of interest may be achieved. The fermentation may also be designed so that undesirable products of metabolism are filtered out or neutralised by the addition of a buffer substance or gegenions suitable for the respective application.

The prepared lipase can be harvested from the fermentation medium. Such a fermentation process is preferable to isolating the lipase from the host cell, i.e. product processing from the cell mass (dry mass), but it requires that suitable host cells or one or more suitable secretion markers or mechanisms and/or transport systems be made available to ensure that the host cells secrete the lipase into the fermentation medium. Without secretion, the lipase may alternatively be isolated from the host cell, i.e. the lipase may be extracted from the cell mass, for example by precipitation with ammonium sulfate or ethanol, or by chromatographic purification.

All of the notes and information presented in the preceding text may be combined in methods for producing lipases as contemplated herein.

A further object of the present disclosure is an agent which contains a lipase as contemplated herein as described previously. The agent preferably has the form of a detergent or cleaning agent.

This object of the present disclosure embraces all conceivable types of detergent or cleaning agent, applicable in either concentrated or diluted form, for use on a commercial scale, in a washing machine or for washing or cleaning by hand. These include for example detergents for textiles, carpets, or natural fibres for which the term detergent is used. These also include for example dishwashing agents for dishwashers or manual dishwashing agents or cleaners for hard surfaces such as metal, glass, china, ceramic, tiles, stone, painted surfaces, plastics, wood or leather, for which the term cleaning agents is used, i.e., besides manual and mechanical dishwashing agents for example scouring agents, glass cleaners, WC fragrance rinses etc. The detergents and cleaning agents within the scope of the present disclosure further extend to include auxiliary washing products which are added to the actual detergent in metered quantities during manual or mechanical fabric washing to obtain additional effect. Detergents and cleaning agents within the scope of the present disclosure also extend to textile pre-treatment and post-treatment substances, that is to say agents with which the item for washing is brought into contact prior to the actual washing process, to soften stubborn soiling, for example, as well as agents which are used in a step following the actual textile washing process to lend the items for washing further desirable properties such as a pleasant feel, freedom from wrinkles or low electrostatic charging. The last group of agents named also includes softeners and the like.

The detergents or cleaning agents as contemplated herein, which may have the form of powdery solids, compressed particles, homogeneous solutions or suspensions, may contain all of the ingredients that are known and usual contents of such agents as well as a lipase as contemplated herein, wherein preferably at least one further ingredient is present in the agent. The agents as contemplated herein may particularly contain surfactants, builders, peroxygen compounds or bleach activators. They may additionally contain water-miscible organic solvents, additional enzymes, sequestering agents, electrolytes, pH regulators and/or additional adjuvants such as optical brighteners, anti-redeposition agents, foam control agents as well as dyes and fragrances and combinations thereof. The detergents or cleaning agents are preferably liquid, i.e. they are flowable at room temperature and under normal pressure (about 20° C. and 1013 mbar).

A combination of a lipase as contemplated herein with one or more further ingredient(s) of the agent is particularly advantageous, since such an agent exhibits improved cleaning power in preferred variants as contemplated herein as a result of synergistic effects created. Particularly the combination of a lipase as contemplated herein with a surfactant and/or a builder and/or a peroxygen compound and/or a bleach activator may engender such a synergy.

Advantageous ingredients of agents as contemplated herein are disclosed in international patent application WO2009/121725, beginning in the penultimate paragraph of page 5 in that document and ending after the second paragraph on page 13, and in WO2012084582 A1, pages 12-27. Reference is herewith expressly made to this disclosure and the contents of the disclosure there is incorporated in the present patent application. In particular, the lipases described herein may be combined advantageously with phosphonates as described on page 4, second paragraph to page 5, first paragraph of WO 2012084582 A1.

An agent as contemplated herein advantageously contains the lipase in a quantity from about 2 μg to about 20 mg, preferably from about 5 μg to about 17.5 mg, particularly preferably from about 20 μg to about 15 mg and most particularly preferably from about 50 μg to about 10 mg per g of the agent. Furthermore, the lipase contained in the agent and/or other ingredients of the agent may be encased in a substance that is impermeable to the enzyme at room temperature or in the absence or water, which substance then becomes perrmeable to the enzyme under the conditions of use of the agent. Such an embodiment of the present disclosure includes the lipase encased in a substance which is impermeable to the enzyme at room temperature or in the absence or water. Additionally, the detergent or cleaning agent itself may also be packed in a receptacle, preferably an air-permeable receptacle, from which it is released shortly before use or during the washing process.

In further embodiments of the present disclosure, the agent

(a) exists in solid form, particularly as a granulated powder having a bulk weight from about 300 g/l to about 1200 g/1, particularly from about 500 g/l to about 900 g/1, or
(b) exists in paste-like or liquid form, and/or
(c) exists in gel-capsule or pouch-like form, and/or
(d) exists as a single component system, or
(e) is divided into multiple components.

These embodiments of the present disclosure comprise all solid, powdered, liquid, gel-like or paste-like dosage forms of agents as contemplated herein, which may optionally also include several such phases and may be present in compressed or non-compressed form. The agent may be present as a granulate powder, particularly with a bulk weight from about 300 g/l to about 1200 g/1, particularly from about 500 g/l to about 900 g/l or from about 600 g/l to about 850 g/1. The solid dosage forms of the agent further include extrudates, granulates, tablets or pouches. Alternatively, the agent may also be in liquid, gel or paste form, for example in the form of a non-aqueous liquid detergents or a non-aqueous paste or in the form of an aqueous liquid detergent or a water-containing paste. In preferred embodiments, the agents are liquid. The agent may also be presented as a single component system. Such agents include a single phase. Alternatively, an agent may include multiple phases. Such an agent is accordingly divided into multiple components.

Detergents or cleaning agents as contemplated herein may contain only one lipase. Alternatively, they may also contain further hydrolytic enzymes or other enzymes in a concentration appropriate to ensure the effectiveness of the agent. A further embodiment of the present disclosure is thus constituted by agents which further comprise one or more additional enzymes. Enzymes that are preferably usable as additional enzymes are all those which demonstrate a catalytic activity in the agent as contemplated herein, particularly a protease, amylase, cellulase, hemicellulase, mannanase, tannase, xylanase, xanthanase, xyloglucanase, β-glucosidase, pectinase, carrageenase, perhydrolase, oxidase, oxidoreductase or other lipases—differentiable from the lipases as contemplated herein—and their mixtures. Further enzymes are contained in the agent, each advantageously in a quantity from about 1×10⁻⁸ to about 5 percent by weight relative to active protein. It is becoming increasingly preferred if each further enzyme is contained in agents as contemplated herein in a quantity from about 1×10⁻⁷-3 percent by weight, from about 0.00001-1 percent by weight, from about 0.00005-0.5 percent by weight, from about 0.0001 to about 0.1 percent by weight and particularly preferably from about 0.0001 to about 0.05 percent by weight relative to active protein. Particularly preferably, the enzymes exhibit synergistic cleaning powers in respect of certain soiling types or stains, i.e., the enzymes in the agent composition strengthen each other's cleaning powers. Most particularly preferably, such a synergy exists between the lipase as contemplated herein contained therein and a further enzyme of an agent as contemplated herein, particularly between said lipase and an amylase and/or a protease and/or a mannanase and/or a cellulase and/or a pectinase. Synergistic effects may arise not only between different enzymes, but also between one or more enzymes and other ingredients of the agent as contemplated herein.

A further object of the present disclosure is a method for cleaning textiles or hard surfaces wherein an agent as contemplated herein is applied in at least one method step, or that a lipase as contemplated herein becomes catalytically active in at least one method step, particularly in such manner that the lipase is used in a quantity from about 40 μg to about 4 g, preferably from about 50 μg to about 3 g, particularly preferably from about 100 μg to about 2 g and most particularly preferably from about 200 μg to about 1 g.

In various embodiments, the method described in the preceding text, the lipase is used at a temperature from about 0-100° C., preferably from about 0-60° C., more preferably from about 20-40° C., most preferably from about 30-40° C. or from about 32-40° C. or approximately about 40° C.

These include both manual and mechanical methods, wherein mechanical methods are preferred. Methods for cleaning textiles generally include successive process steps wherein various active cleaning substances are applied to the item to be cleaned and washed off after the treatment time, or that the item to be cleaned is otherwise treated with a detergent or a solution or dilutation of said agent. Methods for cleaning all materials other than textiles, particularly hard surfaces, involve similar activity. All conceivable washing or cleaning methods can be enhanced by the application of a detergents or cleaning agents as contemplated herein or a lipase as contemplated herein in one of the process steps thereof and therefore represent embodiments of the present disclosure. All information, objects and embodiments described for lipases as contemplated herein and agents that contain them are also applicable to this object of the present disclosure. Accord-ingly, reference is explicitly made here to the relevant place in the disclosure with the note that this disclosure also applies for the preceding methods as contemplated herein.

Since lipases as contemplated herein have hydrolytic activity by their nature and also perform this function in media which otherwise have no cleaning strength, in a simple buffer for example, an individual and/or the only step in such a method may include a lipase as contemplated herein as the only component with active cleaning action brought into contact with the soiling, preferably in a buffer solution or in water. This constitutes a further embodiment of this object of the present disclosure.

Alternative embodiments of this object of the present disclosure also describe methods for treating raw textile materials or caring for textiles in which a lipase as contemplated herein becomes active in at least one step of the method. Of these, methods for raw textile materials, fibres or textiles with natural components are preferred, and most particularly methods for wool or silk.

Finally, the present disclosure also extends to the use of the lipases described herein in detergents or cleaning agents, as described earlier for example, for (improved) removal of soilings that contain grease for example from textiles or hard surfaces.

All information, objects and embodiments described for lipases as contemplated herein and agents that contain them are also applicable to this object of the present disclosure. Accordingly, reference is explicitly made here to the relevant place in the disclosure with the note that this disclosure also applies for the preceding use as contemplated herein.

### EXAMPLES

All molecular biological work steps are carried out in accordance with standard methodologies, such as are described for example in the manual by Fritsch, Sambrook and Maniatis “Molecular cloning: a laboratory manual”, Cold Spring Harbour Laboratory Press, New York, 1989, or comparable pertinent works. Enzymes and kits were used in accordance with the instructions of the respective manufacturers.

Overview of Mutations (Counting Method Conforming to SEQ ID NO:1, i.e. Prosequence+Mature Lipase):

| | | | | | SEQ ID |
|---|---|---|---|---|---|
| Variant | Sequence | | | | NO: |
| Variant 1 | P145L | P260S | H298N | | 2 |
| Variant 2 | V236M | | | | 3 |
| Variant 3 | F311Y | D359G | | | 4 |
| Variant 4 | K169E | | | | 5 |
| Variant 5 | K235N | | | | 6 |
| Variant 6 | S93G | G202V | P308Q | | 7 |
| Variant 7 | H298N | | | | 8 |
| Variant 8 | P145L | P260S | H298N | L156P | 9 |
| Variant 9 | P145L | P260S | H298N | S364F | 10 |
| Variant 10 | P145L | P260S | H298N | Q225H | 11 |
| Variant 11 | P145L | P260S | H298N | P308S | 12 |
| Variant 12 | P145L | P260S | H298N | N328D | 13 |
| Variant 13 | H298N | L156P | | | 14 |
| Variant 14 | H298N | S364F | | | 15 |
| Variant 15 | H298N | Q225H | | | 16 |
| Variant 16 | H298N | P308S | | | 17 |
| Variant 17 | H298N | N328D | | | 18 |
| Variant 18 | H298N | S364F | N193E | | 19 |
| Variant 19 | H298N | S364F | L352T | | 20 |
| Variant 20 | H298N | S364F | N193E | L352T | 21 |

### Example 1: Generation and Characterization of Mutants

The mature part of the lipase gene is mutagenized using error prone mutagenesis according to known methods (“GeneMorph II Random Mutagenesis Kit” from Agilent). The library of mutants is cloned in Pichia pastoris using standard methods. The colonies are inoculated into 96-well deep-well plates and after induction are cultured for a further 72 h. Following centrifuging, the supernatant is examined for lipase activity. Two error prone cycles were carried out consecutively, the first on the wild type lipase, the second on the most effectively stabilised variant from the first cycle (SEQ ID NO:2). Some mutants were also prepared by selective recombination using standard methods.

Activity Assay

In order to identify variants with improved thermal stability, an assay was used on the basis of microtitre plates with para-nitrophenyl-palmitate (p-NPP) as substrate. During enzymatic hydrolysis in the aqueous medium, para-nitrophenolate and palmitate were released and para-nitrophenolate was subsequently detected by absorption measurement at a wavelength of 405 nm. P—NPP is used in the form of an emulsion, it is dissolved beforehand and added to an aqueous buffer containing Na-deoxycholate and alpha olefin sulfonate (AOS) as emulsifiers.

Conditions: pH 8.0, 25° C., 405 nm, measurement time 120 sec with intervals of 30 sec.

Sample buffer in which the lipase supernatants are diluted: 225 mg/mL Brij35, 9 mM CaCl2 and 4.7 mg/mL detergent matrix (see Example 3)
Substrate work buffer: 96.7 mL emulsifier solution (100 mM tris-HCl pH8.0, 6.5 mM deoxycholate, 1.4 g/L AOS)+3.3 mL palmitate solution (7.8 mM p-NPP dissolved in ethanol)

Procedure: Place 20 μL sample diluted in buffer in the MTP and start the reaction by adding 200 μL of the substrate work buffer, shake for 5 sec, start kinetics.

To test the thermal stability of the mutants, the lipase supernatants diluted in the sample buffer were incubated both at 25° C. and at a higher temperature for 40 min before conducting the p-NPP test. The temperature in the first error prone cycle was 32° C. and in the second error prone cycle during screening initially 37° C., then 40° C. during rescreening of the best hits. The factor is formed, Activity after storage at elevated temp. divided by Activity after storage at 25° C. The greater this factor, the better the thermal stability of the lipase variants in detergent matrix.

At least three assays were carried out for each.

Liquid detergent matrix (standard commercial product, without enzymes, opt. brighteners, fragrances or dyes), which was used for the activity test and wash test:

| | Percent by weight | Percent by weight |
|---|---|---|
| | active substance in | active substance in |
| Chemical name | the raw material | the formulation |
| Water demin. | 100 | Rest |
| Alkylbenzene sulfonic acid | 96 | 4.4 |
| Additional anionic surfactants | 70 | 5.6 |
| C12-C18 fatty acids Na-salt | 30 | 2.4 |
| Non-ionic surfactants | 100 | 4.4 |
| Phosphonates | 40 | 0.2 |
| Citric acid | 100 | 1.4 |
| NaOH | 50 | 0.95 |
| Defoaming agents | t.q. | 0.01 |
| Glycerin | 100 | 2.0 |
| Preservatives | 100 | 0.08 |
| Ethanol | 93 | 1.0 |
| Without opt. brightener, | | |
| fragrance, dye and enzymes | | |

### Dosage 4.7 g/L

Results of the thermal stability test and mini wash test:

Error prone cycle 1, ratio of activity 32° C., 40 min/activity 25° C., 40 min:

| Mutant | Activity ratio average |
|---|---|
| Wild type (SEQ ID NO: 1) | 0.27 |
| expressed in Pichia pastoris | |
| Mutant 1 (SEQ ID NO: 2) | 0.95 |
| Mutant 2 (SEQ ID NO: 3) | 0.59 |
| Mutant 3 (SEQ ID NO: 4) | 0.59 |
| Mutant 4 (SEQ ID NO: 5) | 0.56 |
| Mutant 5 (SEQ ID NO: 6) | 0.45 |
| Mutant 6 (SEQ ID NO: 7) | 0.48 |
| Mutant 7 (SEQ ID NO: 8) | 0.38 |

All mutants shows here are more stable than the wild type at 32° C.

Error prone cycle 2 (on Mutant 1), ratio of activity 40° C., 40 min/activity 25° C., 40 min:

| Mutant | Activity ratio average |
|---|---|
| Mutant 1 (Start clone; SEQ ID | 0.26 |
| NO: 2) | |
| Mutant 8 (SEQ ID NO: 9) | 0.40 |
| Mutant 9 (SEQ ID NO: 10) | 0.47 |
| Mutant 10 (SEQ ID NO: 11) | 0.37 |
| Mutant 11 (SEQ ID NO: 12) | 0.35 |
| Mutant 12 (SEQ ID NO: 13) | 0.41 |
| Mutant 14 (SEQ ID NO: 15) | 0.60 |

All mutants shows here are more stable than starter mutant 1 at 40° C. The decisive feature for use in detergents and cleaning agents is the cleaning power of the lipases. This was tested in a mini-wash test on a scale of 1 mL.

Conditions: 40° C., 16° dH water, 1 h incubation

Soilings (from CFT, Center for Testmaterials, Vlaardingen):
CS-61—beef fat colored
The enzymes are used with identical protein with 7.5 mg/washing machine

Place punched out fabric (diameter=10 mm) in microtitre plate, preheat detergent solution to 40° C., final concentration 4.7 g/L, apply liquor and enzyme to the soiling, incubate for 1 h at 20° C. and 40° C. and 600 rpm, then rinse the soiling with clear water several times, allow to dry and determine brightness with a colorimeter. The brighter the fabric becomes, the better the cleaning power is. This measures the L-value=Brightness, the higher the brighter. The test is conducted as triple determination.

The table below shows the delta for the performance of the mutants adjusted by the base performance of the blank (=detergent without lipase).

| | deltaL 20° C. | deltaL 40° C. |
|---|---|---|
| Mutant | (Mutant − Blank) | (Mutant − Blank) |
| Wild type (SEQ ID NO: 1) | 3.5 | n.d. |
| Mutant 1 (SEQ ID NO: 2) | 3.5 | 5.0 |
| Mutant 8 (SEQ ID NO: 9) | 3.7 | 3.1 |
| Mutant 9 (SEQ ID NO: 10) | 4.0 | 5.0 |
| Mutant 11 (SEQ ID NO: 12) | 4.7 | 4.0 |
| Mutant 12 (SEQ ID NO: 13) | 3.3 | 4.1 |

It is evident that mutants perform at least as well as the wild type, in some cases even better. At 40° C. the wild type does not have any washing power due to its instability.

Another washing test was also conducted on a scale of 50 mL:

The washing temperature for the 60 min long main washing cycle was 40° C. and 20° C. The detergent was added in a quantity of 0.2 g in 50 mL water (16° dH). After an incubation period of 60 minutes, the soiling was rinsed several times with clear water, dried, and its brightness was determined with a colorimeter. The brighter the fabric becomes, the better the cleaning power is. This measures the L-value=Brightness, the higher the brighter. The test was conducted as qunituple determination.
The table below shows the delta for the performance of the mutants adjusted by the base performance of the blank (=detergent without lipase).

Soilings (from CFT, Center for Testmaterials, Vlaardingen):

CS-61—beef fat colored
PC-09—pigment/oil

| | deltaL | deltaL | deltaL | deltaL |
|---|---|---|---|---|
| | 20° C. to | 40° C. to | 20° C. to | 40° C. to |
| Mutant | CS-61 | CS-61 | PC-09 | PC-09 |
| Mutant 7 (SEQ ID NO: 8) | 2.5 | 1.3 | 2.3 | 2.8 |
| Mutant 14 (SEQ ID | 1.8 | 2.2 | 1.6 | 2.9 |
| NO: 15) | | | | |

Significant washing power is evident for the mutants at 20° C. and 40° C.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Lipase comprising an amino acid sequence which has the at least about 70% sequence identity with the amino acid sequence specified in SEQ ID NO:1 over the entire length thereof and which has an amino acid substitution in at least one of the position S93, P145, L156, K169, N193, G202, Q225, K235, V236, P260, H298, P308, F311, N328, L352, D359 or S364, referring in each case to the numbering according to SEQ ID NO:1.

2. Lipase according to claim 1, wherein the at least one amino acid substitution is selected from the group of S93G, P145L, L156P, K169E, N193E, G202V, Q225H, K235N, V236M, P260S, H298N, P308S, P308T, F311Y, N328D, L352T, D359G and S364F referring in each case to the numbering according to SEQ ID NO:1.

3. Lipase according to claim 1, wherein it includes an amino acid substitution in position H298 and also includes at least one further amino acid substitution in one of the positions S93, P145, L156, K169, N193, G202, Q225, K235, V236, P260, S308, F311, N328, L352, D359 or S364.

4. Lipase according to claim 1, wherein the lipase includes one of the following amino acid substitution variants:
(i) P145L, P260S and H298N;
(ii) V236M;
(iii) F311Y and D359G;
(iv) K169E;
(v) K235N;
(vi) S93G, G202V and P308Q;
(vii) H298N;
(viii) P145L, P260S, H298N and L156P;
(ix) P145L, P260S, H298N and S364F;
(x) P145L, P260S, H298N and Q225H;
(xi) P145L, P260S, H298N and P308S;
(xii) P145L, P260S, H298N and N328D;
(xiii) H298N and L156P;
(xiv) H298N and S364F;
(xv) H298N and Q225H;
(xvi) H298N and P308S; or
(xvii) H298N and N328D;
(xviii) H298N, S364F and N193E;
(xix) H298N, S364F and L352T;
(xx) H298N, S364F, N193E and L352T;

5. Lipase,
obtained from a lipase according to claim 1 as starting molecule by single or multiple conservative amino acid substitution, wherein the lipase includes at least one of the amino acid substitutions S93G, P145L, L156P, K169E, N193E, G202V, Q225H, K235N, V236M, P260S, H298N, F311Y, N328D, L352T, D359G and S364F in the positions corresponding to positions 93, 145, 156, 169, 193, 202, 225, 235, 236, 260, 298, 308, 311, 328, 352, 359 and 364 according to SEQ ID NO:1.

6. Method for producing a lipase comprising substituting an amino acid in at least one of the positions corresponding to positions 93, 145, 156, 169, 193, 202, 225, 235, 236, 260, 298, 308, 311, 328, 352, 359 and 364 in SEQ ID NO:1 in a starter lipase which has at least about 70% sequence identity with the amino acid sequence specified in SEQ ID NO:1 over the entire length thereof.

7. Method according to claim 6, further comprising one or both of the following method steps:
(a) Inserting a single or multiple conservative amino acid substitution, wherein the lipase includes at least one of the amino acid substitutions S93G, P145L, L156P, K169E, N193E, G202V, Q225H, K235N, V236M, P260S, H298N, P308S, P308T, F311Y, N328D, L352T, D359G or S364F in the positions corresponding to positions 93, 145, 156, 169, 193, 202, 225, 235, 236, 260, 298, 308, 311, 328, 352, 359 and 364 according to SEQ ID NO:1; and
b) Modifying the amino acid sequence by fragmentation, deletion, insertion or substitution mutagenesis in such manner that the lipase includes an amino acid sequence which matches the starting molecule over a length of at least about 50 consecutive amino acids, wherein the lipase at least one of the amino acid substitutions S93G, P145L, L156P, K169E, N193E, G202V, Q225H, K235N, V236M, P260S, H298N, P308S, P308T, F311Y, N328D, L352T, D359G or S364F in the positions corresponding to positions 93, 145, 156, 169, 193, 202, 225, 235, 236, 260, 298, 308, 311, 328, 352, 359 and 364 according to SEQ ID NO:1.

8. (canceled)

9. (canceled)

10. (canceled)

11. (canceled)

12. (canceled)

13. (canceled)

14. (canceled)

15. Lipase according to claim 1, wherein it includes an amino acid substitution in position H298N, and also includes at least one further amino acid substitution from the group consisting of S93G, P145L, L156P, K169E, N193E, G202V, Q225H, K235N, V236M, P260S, P308S, P308T, F311Y, N328D, L352T, D359G and S364F referring in each case to the numbering according to SEQ ID NO:1.

16. Method according to claim 6 comprising substituting an amino acid in at least one of the positions corresponding to positions 93, 145, 156, 169, 193, 202, 225, 235, 236, 260, 298, 308, 311, 328, 352, 359 and 364 in SEQ ID NO:1 in a starter lipase which has at least about 70% sequence identity with the amino acid sequence specified in SEQ ID NO:1 over the entire length thereof in such manner that the lipase includes the acid substitution S93G, P145L, L156P, K169E, N193E, G202V, Q225H, K235N, V236M, P260S, H298N, P308S, P308T, F311Y, N328D, L352T, D359G or S364F in at least one position.

17. Method according to claim 6, further comprising both of the following method steps:
(a) Inserting a single or multiple conservative amino acid substitution, wherein the lipase includes at least one of the amino acid substitutions S93G, P145L, L156P, K169E, N193E, G202V, Q225H, K235N, V236M, P260S, H298N, P308S, P308T, F311Y, N328D, L352T, D359G or S364F in the positions corresponding to positions 93, 145, 156, 169, 193, 202, 225, 235, 236, 260, 298, 308, 311, 328, 352, 359 and 364 according to SEQ ID NO:1;
b) Modifying the amino acid sequence by fragmentation, deletion, insertion or substitution mutagenesis in such manner that the lipase includes an amino acid sequence which matches the starting molecule over a length of at least about 365 consecutive amino acids, wherein the lipase at least one of the amino acid substitutions S93G, P145L, L156P, K169E, N193E, G202V, Q225H, K235N, V236M, P260S, H298N, P308S, P308T, F311Y, N328D, L352T, D359G or S364F in the positions corresponding to positions 93, 145, 156, 169, 193, 202, 225, 235, 236, 260, 298, 308, 311, 328, 352, 359 and 364 according to SEQ ID NO:1.

18. A Lipase obtained from a lipase according to claim 1 as starting molecule by fragmentation, deletion, insertion or substitution mutagenesis and comprises an amino acid sequence which matches the starting molecule over a length of at least about 50 consecutive amino acids, wherein the lipase comprises at least one of the amino acid substitutions S93G, P145L, L156P, K169E, N193E, G202V, Q225H, K235N, V236M, P260S, H298N, P308S, P308T, F311Y, N328D, L352T, D359G or S364F in the positions corresponding to positions 93, 145, 156, 169, 193, 202, 225, 235, 236, 260, 298, 308, 311, 328, 352, 359 and 364 according to SEQ ID NO:1.

19. A Lipase obtained from a lipase according to claim 1 as starting molecule by fragmentation, deletion, insertion or substitution mutagenesis and comprises an amino acid sequence which matches the starting molecule over a length of at least about 365 consecutive amino acids, wherein the lipase comprises at least one of the amino acid substitutions S93G, P145L, L156P, K169E, N193E, G202V, Q225H, K235N, V236M, P260S, H298N, P308S, P308T, F311Y, N328D, L352T, D359G or S364F in the positions corresponding to positions 93, 145, 156, 169, 193, 202, 225, 235, 236, 260, 298, 308, 311, 328, 352, 359 and 364 according to SEQ ID NO:1.
